(19) United States
(12) Patent Application Publication
(10) Pub. No.: US 2025/0135059 A1
(43) Pub. Date: May 1, 2025

(54) COMPOSITIONS COMPRISING FRAGRANCE MATERIALS

(71) Applicant: The Procter & Gamble Company, Cincinnati, OH (US)

(72) Inventors: Bernardo M. AGUILERA-MERCADO, Kenwood, OH (US); Philip John PORTER, Mason, OH (US); Mu WANG, Cincinnati, OH (US); Ryan Johnie SMITH, Cincinnati, OH (US); Musen Zhou, Montgomery, OH (US); Michael Patrick HAYES, Clarksville, OH (US); Karen Hussong STRADER, Springboro, OH (US); Fabrizio MELI, Montgomery, OH (US)

(21) Appl. No.: 18/926,975

(22) Filed: Oct. 25, 2024

Related U.S. Application Data
(60) Provisional application No. 63/593,280, filed on Oct. 26, 2023.

(51) Int. Cl. A61L 9/012; A61K 8/37; A61L 9/04; A61Q 13/00; C11B 9/00; C11D 3/20; C11D 3/50

(52) U.S. Cl. CPC A61L 9/012; A61K 8/37; A61L 9/044; A61Q 13/00; C11B 9/0019; C11B 9/003; C11B 9/0053; C11D 3/2093; C11D 3/50; A61K 2800/5922; C11D 2111/12; C11D 2111/14

(57) ABSTRACT

Compositions, such as consumer products and/or fragrance compositions, that include a fragrance material that has fruity notes.

### FIELD OF THE INVENTION

The present disclosure relates to compositions, such as consumer products and/or fragrance compositions, that include fragrance materials with fruity notes. The present disclosure further relates to processes of making and using such compositions.

### BACKGROUND OF THE INVENTION

Consumer products that incorporate a pleasing scent, such as cleaning products, soaps, detergents, fabric fresheners, antiperspirants, air fresheners, and fine fragrances, are always in high demand. Consumers often prefer fragrance materials having an odor that is described as “fruity” or as having “fruity notes.” Most fragrance materials with fruity notes possess an ester functional group. However, a significant disadvantage to most ester-possessing fruity fragrance materials is a relatively low vapor pressure. A low vapor pressure fragrance material resists escape from a liquid consumer product composition, and therefore has a low probability of reaching the consumer's nose and imparting delight. As such, there is a continuing need for new fragrance materials that have both fruity notes and a relatively high vapor pressure.

Moreover, in order to overcome the low vapor pressure and still impart the desired fruity notes to a consumer product, a manufacturer would traditionally just add more ester-possessing fruity fragrance material into the consumer product. New fragrance materials that have both fruity notes and a relatively high vapor pressure will allow dosage at lower levels in a consumer product, therefore providing a more sustainable consumer product that also provides the desired fruity notes.

Accordingly, there is a continuing need for new fragrance materials with fruity notes that can be utilized in consumer products, compositions, and related processes.

### SUMMARY OF THE INVENTION

The present disclosure relates to compositions, such as consumer products and/or fragrance compositions, that include a fragrance material including at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.

For example, the present disclosure relates to a consumer product that includes: a first fragrance material that includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, where the fragrance material is present in an amount of at least 0.0003%, by weight of the consumer product composition, and a treatment adjunct.

The present disclosure also relates to consumer products, such as a laundry detergent and other cleaning products, that include: a first fragrance material that includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, where the fragrance material is present in an amount between about 0.0003% and about 0.8%, by weight of the consumer product composition, and a treatment adjunct.

The present disclosure also relates to consumer products, such as fine fragrance compositions, laundry scent boosters, and air-freshener liquid compositions, that include: a first fragrance material that includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, where the fragrance material is present in an amount of between about 0.0003% and about 20.0%, by weight of the consumer product composition, and a treatment adjunct.

The present disclosure also relates to a process of making a consumer product, the process including the steps of combining a first fragrance material with a treatment adjunct or a substrate, where the first fragrance material includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.

The present disclosure also relates to a process of treating a surface or an environment, the process including the step of providing a fragrance material that includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, to a surface or environment.

The present disclosure also relates to a use of a fragrance material that includes at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate on a surface or an environment.

### DETAILED DESCRIPTION OF THE INVENTION

The present disclosure relates to compositions, consumer products, and processes that include fragrance materials with fruity notes. In addition to having fruity notes, the ester-containing fragrance materials detailed herein also have certain advantageous characteristics that can make them more attractive to consumer product manufacturers. For example, because the ester-containing fragrance materials described herein have a relatively higher vapor pressures compared with traditional fragrance materials with fruity notes, the ester-containing fragrance materials detailed herein may be even more preferable by saving on raw material costs, carbon footprint and/or formulation space. Moreover, because the fragrance materials detailed herein contain an ester bond, such fragrance materials would also be expected to have improved biodegradability.

The fragrance materials, compositions, consumer products, and related processes are discussed in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein, “consumer products” can include baby care, beauty care, fabric & home care, family care, feminine care, health care, food (including snack), beverage, and/or fine fragrance products or devices generally intended to be used or consumed in the form in which it is sold. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use including fine fragrances; and shaving products, products for and/or methods relating to treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition; food products, including processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); beverages, including carbonated beverages, tea, flavored water, and coffee; and water purification.

As used herein the phrase “fabric care product” includes compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation.

Unless otherwise noted, all component or composition levels are in reference to the active portion of that component or composition, and are exclusive of impurities, for example, residual solvents or by-products (including minor decomposition products that may form upon storage), which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Ester Containing Fragrance Materials with Fruity Notes:

Many of the compositions, products, and processes described herein include an ester-containing fragrance material with fruity notes. As described in more detail below, it is believed that such materials can provide a pleasing scent to compositions and products while also having certain advantageous characteristics, such as a relatively higher vapor pressure that provides a more noticeable bloom of the fragrance, which facilitate savings in raw material costs and a lower carbon footprint.

As described herein, the particular detailed ester-containing compounds are referred to as fragrance materials. As described further in the Examples section below, certain interesting ester-containing compounds such as, but not limited to, 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate are characterized by having fruity notes, as well as certain other desirable characteristics, including relatively high vapor pressure (when compared to other ester-containing fragrance materials with fruity notes), that may make them desirable replacements for traditional fragrance materials with fruity notes in consumer products. These 4 interesting fragrance materials that have proven useful as detailed herein are listed in Table 1 below.

TABLE 1

| | | **Vapor Pressure |
|---|---|---|
| Name | CAS | @ 25° C. Torr |
| 3-Methoxybutyl acetate | 4435-53-4 | 1.158 |
| Hexyl methacrylate | 142-09-6 | 2.213 |
| Diethyl isobutylmalonate | 10203-58-4 | 0.0167 |
| Diethyl cyclobutane-1,1-dicarboxylate | 3779-29-1 | 0.0907 |
| methyl 4-methoxybicyclo[2.2.2]octane- | 81687-90-3 | 0.0433 |
| 1-carboxylate | | |

**Vapor Pressure @ 25° C. is a theoretical value predicted by a Physicochemical Properties model simulator from ACD/Labs. This vapor pressure model is accepted industry wide and used by CAS/SciFinder. Calculate Physicochemical Properties | PhysChem Suite | ACD/Labs (acdlabs.com)

The lowest vapor pressure predicted for the fragrance materials listed in Table 1 (diethyl isobutylmalonate) is higher than 65% of all known fruity ester perfume raw materials' predicted vapor pressures. Accordingly, all four of the listed fragrance materials have relatively high vapor pressures when compared to all known fruity ester PRMs. This prediction of a relatively high vapor pressure, leading to a higher probability of reaching the consumer's nose and imparting delight, will further be proven using Head Space data in the Examples section herein.

### Consumer Products

The present disclosure relates to consumer products that comprise an ester containing fragrance material with a fruity note. Typically, the consumer products will also comprise a treatment adjunct. Additionally, or alternatively, the consumer product may comprise a fragrance material that is provided to a substrate.

The consumer product may take any suitable form. Preferably, the consumer product is a baby care product, a beauty care product, a fabric care product, a home care product, a family care product, a feminine care product, a health care product, a food product, a beverage product, a fine fragrance product, or a combination thereof. The consumer products of the present disclosure may be compositions, devices, or combinations thereof that are useful in baby care, beauty care, fabric care, home care, family care, feminine care, health care, food, beverage, and/or fine fragrance applications. For consumer health reasons, it may be preferred that the consumer product is not a tobacco-related product, such as cigarettes, e-cigarettes, or products (such as aerosols) intended to give a tobacco-like aroma.

The consumer product may preferably be a fabric care product and/or a home care product, wherein the home care product is selected from the group consisting of a hard surface cleaner, a dish care product, an air care product, or a combination thereof. The consumer product may be a cleaning and/or treatment product.

The consumer product may take any suitable form or combination of forms. For example, the consumer product may comprise a form selected from a liquid composition, a granular composition, a hydrocolloid, a single-compartment pouch, a multi-compartment pouch, a dissolvable sheet, a pastille or bead, a fibrous article, a tablet, a stick, a bar, a flake, a spray, a foam/mousse, a non-woven sheet, a dispensing device, a cream, an absorbent article, a paste, an adhesive, an adhesive patch, or a mixture thereof. The consumer product may be a composition, such as a liquid composition, that is housed in a container, dispensing device, or other suitable article.

The consumer product may comprise a first fragrance material, wherein the fragrance material is present in an amount of at least 0.0003%, by weight of the consumer product, and a treatment adjunct.

The consumer product may comprise the first fragrance material (e.g., an ester-containing fragrance material that has fruity notes) in an amount of at least 0.0003%, or at least 0.0005%, or at least 0.001%, or at least 0.01%, or at least 0.05%, or at least 0.5%, or at least 0.8%, or at least 1.0%, or at least 2.0%, or at least 5.0%, or at least 10.0%, by weight of the consumer product. The consumer product may comprise the first fragrance material (e.g., an ester-containing fragrance material that has fruity notes) in an amount of between about 0.0003% and about 0.8%, by weight of the consumer product. Consumer products with such ranges of the first fragrance material are typically laundry products, detergents and other cleaning products that contain a surfactant. For consumer products containing high amounts of perfume, such as fine fragrance products, scent boosters, energized air fresheners, and volatile composition dispensers (energized or not), it may be preferable for such consumer products to comprise the first fragrance material (e.g., an ester-containing fragrance material that has fruity notes) in an amount of between about 0.0003% and about 20.0%, by weight of the consumer product. It is preferred to have a certain minimum amount of the fragrance material present in order to deliver the desired olfactory performance.

In some embodiments, the consumer product may comprise the first fragrance material (e.g., an ester-containing fragrance material that has fruity notes) in an amount of up to about 99%, or up to an amount of about 50%, or up to about 25%, or up to about 10%, or up to about 5%, or up to about 1%, by weight of the consumer product. It may be preferred to have a certain maximum amount or limit of the first fragrance material present in order to include other adjuncts or fragrance materials.

The consumer product may comprise the first fragrance material (e.g., an ester-containing fragrance material that has fruity notes) in an amount of from about 0.01% to about 99%, or from about 0.1% to about 50%, or from about 0.5% to about 10%, or from about 1% to about 5%, by weight of the consumer product.

The consumer product may comprise the first fragrance material (e.g., an ester-containing fragrance material that has fruity notes) and a second fragrance material selected from the group consisting of any fragrance material that is suitable for the intended product or end use, wherein the total fragrance material is made up of least 10%, or at least 20%, or at least 30%, or at least 40%, or at least 50%, or at least 60%, or at least 70%, or at least 80%, or at least 90%, or at least 95% biodegradable materials. The term “biodegradable” is defined herein as chemical dissolution, disintegration, or digestion of biodegradable perfume by bacteria or other biological means at a rate above 50% according to the OECD301 C test method, which defines readily biodegradability of materials. In this test the biodegradable perfumes are suspended in a phosphate buffered media containing an activated sludge inoculum and the consumption of oxygen and or the formation of carbon dioxide is measured via an electrolytic respirometer. The test substance is the sole carbon and energy source and under aerobic conditions microorganisms metabolize organic substances producing CO2 as the ultimate product.

For example, the consumer product may comprise: a first fragrance material that is an ester-containing fragrance material with fruity notes; an optional second fragrance material selected from the group detailed in Table 2 herein, and mixtures thereof; and a treatment adjunct; wherein the weight ratio of the first fragrance material to the second fragrance material, if present, is from 100:0 to 1:99.

The weight ratio of the first fragrance material (e.g., an ester-containing fragrance material that has fruity notes) to the second fragrance material selected from the group detailed in Table 2 herein, and mixtures thereof may be from 100:0 to 1:99, or from 100:0 to 25:75, or from 100:0 to 50:50, or from 100:0 to 51:49, or from 100:0 to 75:25, or from 100:0 to 90:10, or from 100:0 to 95:5, or from 100:0 to 99:1.

It may be preferred to formulate only one or more of the presently disclosed materials in Table 1 and to leave out other fragrance materials (e.g., the fragrance materials listed in Table 2). For example, the consumer product may be substantially free of a second fragrance material such as the fragrance materials listed in Table 2. In particular, a consumer product may comprise ester-containing fragrance materials with fruity notes as detailed in Table 1 and be substantially free of the fragrance materials as detailed in Table 2.

The first fragrance material may be present as a free oil in the consumer product, or it may be part of a perfume delivery system. For example, the fragrance materials with fruity notes detailed herein may be encapsulated, or otherwise associated with (e.g., embedded in), a delivery particle. For example, fragrance materials with fruity notes may be in the core of a core-shell delivery particle. The fragrance materials with fruity notes may be covalently or ionically bonded, preferably covalently bonded, to a carrier material, thereby forming a pro-fragrance material that releases the fragrance material when the bond is broken, such as through exposure to water or light. Thus, the fragrance material, e.g., Table 1 fragrance materials, may be part of a perfume delivery system, where the perfume delivery system is a delivery particle, a pro-fragrance material, or a combination thereof.

As described above, the consumer product comprises a treatment adjunct. The treatment adjunct material may provide a benefit in the intended end-use of a composition, or it may be a processing and/or stability aid. The precise nature of the treatment adjunct(s), and levels of incorporation thereof, will depend on the physical form of the composition and the nature of the operation for which it is to be used. One of ordinary skill will be able to select appropriate materials and levels for a given product or application.

Suitable treatment adjuncts may include antioxidants, surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, or mixtures thereof.

The treatment adjunct may comprise a carrier. Such carriers may be hydrophilic (e.g., water) or hydrophobic (e.g., an oil) in nature. The presently described fragrance materials with fruity notes are relatively hydrophobic in nature and may be conveniently coupled with a hydrophobic carrier. Particularly useful carriers in consumer products include, but are not limited to, isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or mixtures thereof. Thus, the consumer product may comprise a carrier, where the carrier is preferably IPM, DPM, or mixtures thereof, Preferably, the carrier comprises isopropyl myristate.

Isopropyl myristate, as well as other hydrophobic carriers, may be particularly useful when the fragrance materials with fruity notes are encapsulated in certain core-shell delivery particles, such as those having acrylate-based or chitosan-based shells. The cores of the delivery particles may comprise both the isopropyl myristate and the fragrance materials with fruity notes.

### Fragrance Composition

The present disclosure also relates to fragrance compositions. Such compositions typically comprise mixtures of fragrance ingredients. The fragrance compositions of the present disclosure may take the form of premixes or intermediate compositions that are combined with other materials to make final products, such as consumer products.

For example, the fragrance composition may take the form of a fragrance composition that is a mixture of fragrance materials, such as a perfume accord that may be formulated into a consumer product.

The fragrance composition may comprise a first fragrance material and a second fragrance material that is different than the first fragrance material, where the first fragrance material is a fragrance material as detailed in Table 1, and where the first fragrance material is present in an amount of at least 1%, by weight of the fragrance composition.

The first fragrance material may be present in an amount of at least 0.005%, or at least 0.05%, or even 0.1% by weight of the fragrance composition. The first fragrance material may be present in an amount of from about 0.005% to about 20%, or from about 0.05% to about 10%, or from about 0.005% to about 3%, or from about 0.1% to about 1.5% by weight of the fragrance composition. The first fragrance material may be present in an amount of at least 1%, or at least 2%, or at least 5%, or even at least 10%, or even at least 20%, by weight of the fragrance composition. The first fragrance material may be present in an amount of from 1% to about 99%, or from 1% to about 50%, or from about 1% to about 25%, or from about 1% to about 10%, or from about 1% to about 5%, by weight of the fragrance composition.

The second fragrance material may be selected from the group consisting of any fragrance material that is suitable for the intended product or end use (e.g., one or more of the fragrance materials detailed in Table 2). The term “perfume raw material” (or “PRM”) as used herein refers to compounds having a molecular weight of at least about 100 g/mol and which are useful in imparting an odor, fragrance, essence, or scent, either alone or with other perfume raw materials. Typical PRMs comprise inter alia aromatic and/or aliphatic alcohols, ketones, aldehydes, esters, ethers, carboxylic acids, nitriles, nitrites and alkenes, such as terpene, and alkynes. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

As non-limiting, illustrative examples, additional suitable second fragrance materials are provided below in Table 2. The second fragrance material may be selected from the group provided in Table 2, or combinations thereof.

TABLE 2

| NAME | CAS | IUPAC NAME |
|---|---|---|
| ALLYL AMYL | 67634-00-8 | ALLYL 2-(ISOPENTYLOXY)ACETATE |
| GLYCOLATE | | |
| ALLYL CAPROATE | 123-68-2 | ALLYL HEXANOATE |
| ALLYL CYCLOHEXANE | 2705-87-5 | ALLYL 3-CYCLOHEXYLPROPANOATE |
| PROPIONATE | | |
| ALLYL HEPTOATE | 142-19-8 | ALLYL HEPTANOATE |
| ALLYL PHENOXY | 7493-74-5 | ALLYL 2-PHENOXYACETATE |
| ACETATE | | |
| AMYL ACETATE | 123-92-2 | ISOPENTYL ACETATE |
| ANAPEAR 8753453 | 189440-77-5 | METHYL OCTA-4,7-DIENOATE |
| ANISYL ACETATE | 104-21-2 | 4-METHOXYBENZYL ACETATE |
| BENZYL ACETATE | 140-11-4 | BENZYL ACETATE |
| BENZYL BUTYRATE | 103-37-7 | BENZYL BUTYRATE |
| BENZYL ISO BUTYRATE | 103-28-6 | BENZYL ISOBUTYRATE |
| BENZYL PROPIONATE | 122-63-4 | BENZYL PROPIONATE |
| BUTYL ACETATE | 123-86-4 | BUTYL ACETATE |
| BUTYL LACTATE | 138-22-7 | BUTYL 2-HYDROXYPROPANOATE |
| CARVYL ACETATE | 97-42-7 | 2-METHYL-5-(PROP-1-EN-2- |
| | | YL)CYCLOHEX-2-EN-1-YL ACETATE |
| CIS 3 HEXENYL ACETATE | 3681-71-8 | HEX-3-EN-1-YL ACETATE |
| CIS 3 HEXENYL | 16491-36-4 | HEX-3-EN-1-YL BUTYRATE |
| BUTYRATE | | |
| CIS-3-HEXENYL ALPHA | 10094-41-4 | HEX-3-EN-1-YL 2-METHYLBUTANOATE |
| METHYL BUTYRATE | | |
| CIS-3-HEXENYL | 25152-85-6 | HEX-3-EN-1-YL BENZOATE |
| BENZOATE | | |
| CIS-3-HEXENYL CIS-3- | 61444-38-0 | HEX-3-EN-1-YL HEX-3-ENOATE |
| HEXENOATE | | |
| CITRONELLYL ACETATE | 67601-05-2 | 3,7-DIMETHYLOCT-6-EN-1-YL ACETATE |
| CONIFERAN | 67874-72-0 | 2-(TERT-PENTYL)CYCLOHEXYL |
| | | ACETATE |
| COSMOFRUIT | 1370711-06-0 | 2-METHYLPENTAN-3-YL BUT-2- |
| | | ENOATE |
| CYCLABUTE | 67634-20-2 | 3A,4,5,6,7,7A-HEXAHYDRO-1H-4,7- |
| | | METHANOINDEN-5-YL ISOBUTYRATE |
| CYCLO GALBANATE | 68901-15-5 | ALLYL 2-(CYCLOHEXYLOXY)ACETATE |
| CYCLOBUTANATE | 113889-23-9 | CYCLOBUTANATE |
| CYCLOHEXYL ETHYL | 21722-83-8 | 2-CYCLOHEXYLETHYL ACETATE |
| ACETATE | | |
| DELTA DECALACTONE | 705-86-2 | 6-PENTYLTETRAHYDRO-2H-PYRAN-2- |
| | | ONE |
| DELTA DODECALACTONE | 713-95-1 | 6-HEPTYLTETRAHYDRO-2H-PYRAN-2- |
| | | ONE |
| DELTA OCTALACTONE | 698-76-0 | 6-PROPYLTETRAHYDRO-2H-PYRAN-2- |
| FCC | | ONE |
| DELTA-UNDECALACTONE | 710-04-3 | 6-HEXYLTETRAHYDRO-2H-PYRAN-2- |
| FCC | | ONE |
| DIETHYL MALONATE | 105-53-3 | DIETHYL MALONATE |
| DIMETHYL | 85-91-6 | METHYL 2- |
| ANTHRANILATE | | (METHYLAMINO)BENZOATE |
| DIMETHYL BENZYL | 151-05-3 | 2-METHYL-1-PHENYLPROPAN-2-YL |
| CARBINYL ACETATE | | ACETATE |
| DIMETHYL BENZYL | 10094-34-5 | 2-METHYL-1-PHENYLPROPAN-2-YL |
| CARBINYL BUTYRATE | | BUTYRATE |
| ESTERLY | 3289-28-9 | ETHYL CYCLOHEXANECARBOXYLATE |
| ETHYL 2 METHYL | 39255-32-8 | ETHYL 2-METHYLPENTANOATE |
| PENTANOATE | | |
| ETHYL 2-TRANS-4-CIS- | 3025-30-7 | ETHYL DECA-2,4-DIENOATE |
| DECADIENOATE | | |
| ETHYL ACETATE | 141-78-6 | ETHYL ACETATE |
| ETHYL ACETOACETATE | 141-97-9 | ETHYL 3-OXOBUTANOATE |
| ETHYL BENZOATE | 93-89-0 | ETHYL BENZOATE |
| ETHYL BUTYRATE | 105-54-4 | ETHYL BUTYRATE |
| ETHYL CAPROATE | 123-66-0 | ETHYL HEXANOATE |
| ETHYL ISOBUTYRATE | 97-62-1 | ETHYL ISOBUTYRATE |
| ETHYL LACTATE | 97-64-3 | ETHYL 2-HYDROXYPROPANOATE |
| ETHYL METHYL PHENYL | 77-83-8 | ETHYL 3-METHYL-3-PHENYLOXIRANE- |
| GLYCIDATE | | 2-CARBOXYLATE |
| ETHYL OENANTHATE | 106-30-9 | ETHYL HEPTANOATE |
| ETHYL PHENYL | 121-39-1 | ETHYL 3-PHENYLOXIRANE-2- |
| GLYCIDATE | | CARBOXYLATE |
| ETHYL PROPIONATE | 105-37-3 | ETHYL PROPIONATE |
| ETHYL SAFRANATE | 35044-59-8 | ETHYL 2,6,6-TRIMETHYLCYCLOHEXA- |
| | | 1,3-DIENE-1-CARBOXYLATE |
| ETHYL-2-METHYL | 7452-79-1 | ETHYL 2-METHYLBUTANOATE |
| BUTYRATE | | |
| ETHYL-3,5,5-TRIMETHYL- | 67707-75-9 | ETHYL 3,5,5-TRIMETHYLHEXANOATE |
| HEXANOATE | | |
| FRUCTALATE | 72903-27-6 | DIETHYL CYCLOHEXANE-1,4- |
| | | DICARBOXYLATE |
| FRUITATE | 80657-64-3 | ETHYL OCTAHYDRO-3AH-4,7- |
| | | METHANOINDENE-3A-CARBOXYLATE |
| FRUTENE | 68912-13-0 | TRICYCLODECENYL PROPIONATE |
| GAMMA DECALACTONE | 706-14-9 | 5-HEXYLDIHYDROFURAN-2(3H)-ONE |
| GAMMA | 2305-05-7 | GAMMA DODECALACTONE |
| DODECALACTONE | | |
| GAMMA NONALACTONE | 104-61-0 | 5-PENTYLDIHYDROFURAN-2(3H)-ONE |
| (EX. CASTOR OIL) | | |
| GAMMA OCTALACTONE | 104-50-7 | 5-BUTYLDIHYDROFURAN-2(3H)-ONE |
| GERANYL ACETATE | 105-87-3 | 3,7-DIMETHYLOCTA-2,6-DIEN-1-YL |
| | | ACETATE |
| GERANYL BUTYRATE | 106-29-6 | 3,7-DIMETHYLOCTA-2,6-DIEN-1-YL |
| | | BUTYRATE |
| HEXYL ACETATE | 142-92-7 | HEXYL ACETATE |
| HEXYL CAPROATE | 6378-65-0 | HEXYL HEXANOATE |
| HEXYL ISOBUTYRATE | 2349-07-7 | HEXYL ISOBUTYRATE |
| HEXYL-2-METHYL | 10032-15-2 | HEXYL 2-METHYLBUTANOATE |
| BUTYRATE | | |
| ISO NONYL ACETATE | 58430-94-7 | 3,5,5-TRIMETHYLHEXYL ACETATE |
| ISO PENTYRATE | 80118-06-5 | 4-METHYLPENT-4-EN-2-YL |
| | | ISOBUTYRATE |
| ISO PROPYL 2-METHYL- | 66576-71-4 | ISOPROPYL 2-METHYLBUTANOATE |
| BUTYRATE | | |
| ISOAMYL BUTYRATE | 106-27-4 | ISOPENTYL BUTYRATE |
| ISOAMYL PROPIONATE | 105-68-0 | ISOPENTYL PROPIONATE |
| JAMUNATE | 7605-52-9 | METHYL 3-METHYLCYCLOHEXANE-1- |
| | | CARBOXYLATE |
| JASMOLACTONE | 32764-98-0 | 6-(PENT-3-EN-1-YL)TETRAHYDRO-2H- |
| | | PYRAN-2-ONE |
| KOUMALACTONE | 92015-65-1 | 3,6- |
| | | DIMETHYLHEXAHYDROBENZOFURAN- |
| | | 2(3H)-ONE |
| LACTOJASMON | 7011-83-8 | 5-HEXYL-5-METHYLDIHYDROFURAN- |
| | | 2(3H)-ONE |
| LINALYL ISO BUTYRATE | 78-35-3 | 3,7-DIMETHYLOCTA-1,6-DIEN-3-YL |
| | | ISOBUTYRATE |
| MALTOL ISOBUTYRATE | 65416-14-0 | 2-METHYL-4-OXO-4H-PYRAN-3-YL |
| | | ISOBUTYRATE |
| MALTOL PROPIONATE | 68555-63-5 | 2-METHYL-4-OXO-4H-PYRAN-3-YL |
| | | PROPIONATE |
| METHYL ANTHRANILATE | 134-20-3 | METHYL 2-AMINOBENZOATE |
| METHYL BENZOATE | 93-58-3 | METHYL BENZOATE |
| METHYL CINNAMATE | 103-26-4 | METHYL 3-PHENYLACRYLATE |
| METHYL DIOXOLAN | 6413-10-1 | ETHYL 2-(2-METHYL-1,3-DIOXOLAN-2- |
| | | YL)ACETATE |
| METHYL OCTALACTONE | 39212-23-2 | 5-BUTYL-4-METHYLDIHYDROFURAN- |
| | | 2(3H)-ONE |
| METHYL PHENYL | 93-92-5 | 1-PHENYLETHYL ACETATE |
| CARBINYL ACETATE | | |
| METHYL-2-NONENOATE | 111-79-5 | METHYL NON-2-ENOATE |
| MONTAVERDI | 188570-78-7 | HEX-3-EN-1-YL |
| | | CYCLOPROPANECARBOXYLATE |
| NATURAL ISOBUTYL | 110-19-0 | ISOBUTYL ACETATE |
| ACETATE | | |
| NONALACTONE | 63357-97-1 | 5-PENTYLDIHYDROFURAN-2(3H)-ONE |
| OCTAHYDRO COUMARIN | 4430-31-3 | OCTAHYDRO-2H-CHROMEN-2-ONE |
| PHENOXY ETHYL ISO | 103-60-6 | 2-PHENOXYETHYL ISOBUTYRATE |
| BUTYRATE | | |
| PHENYL ETHYL ISO | 103-48-0 | PHENETHYL ISOBUTYRATE |
| BUTYRATE | | |
| PIVACYCLENE | 68039-44-1 | 3A,4,5,6,7,7A-HEXAHYDRO-1H-4,7- |
| | | METHANOINDEN-6-YL PIVALATE |
| PRENYL ACETATE | 1191-16-8 | 3-METHYLBUT-2-EN-1-YL ACETATE |
| PULPANAT | 76649-26-8 | NONA-3,6-DIEN-1-YL ACETATE |
| ROMASCONE | 81752-87-6 | METHYL 2,2-DIMETHYL-6- |
| | | METHYLENECYCLOHEXANE-1- |
| | | CARBOXYLATE |
| ROSEOLATE 980310 | 1584709-99-8 | 1-ISOPROPOXY-1-OXOPROPAN-2-YL |
| | | PIVALATE |
| THESARON | 22471-55-2 | ETHYL 2,2,6- |
| | | TRIMETHYLCYCLOHEXANE-1- |
| | | CARBOXYLATE |
| TRIACETIN | 25395-31-7 | GLYCEROL DIACETATE |
| TROPICALIA | 10138-32-6 | ETHYL BICYCLO[2.2.1]HEPT-5-ENE-2- |
| | | CARBOXYLATE |
| UNDECALACTONE | 104-67-6 | 5-HEPTYLDIHYDROFURAN-2(3H)-ONE |
| VELBERRY | 13431-57-7 | 2,2-DIMETHYLPROPANE-1,3-DIYL |
| | | DIACETATE |
| VERDOX | 88-41-5 | 2-(TERT-BUTYL)CYCLOHEXYL |
| | | ACETATE |
| 2,8-EPITHIO-CIS-P- | 68398-18-5 | 4,7,7-TRIMETHYL-6- |
| MENTHANE | | THIABICYCLO[3.2.1]OCTANE |
| 2-CYCLOPENTYL | 4884-24-6 | [1,1′-BI(CYCLOPENTAN)]-2-ONE |
| CYCLOPENTANONE | | |
| 2-ISOBUTYLTHIAZOLE | 18640-74-9 | 2-ISOBUTYLTHIAZOLE |
| 2-ISOPROPYL-4-METHYL | 15679-13-7 | 2-ISOPROPYL-4-METHYLTHIAZOLE |
| THIAZOLE | | |
| 3,6-NONADIEN-1-OL | 76649-25-7 | NONA-3,6-DIEN-1-OL |
| 4-METHYL-4- | 19872-52-7 | 4-MERCAPTO-4-METHYLPENTAN-2- |
| MERCAPTOPENTAN-2- | | ONE |
| ONE | | |
| ACALEA TBHQ | 84697-09-6 | 2-(4- |
| | | METHYLBENZYLIDENE)HEPTANAL |
| ACETOPHENONE | 98-86-2 | ACETOPHENONE |
| ALPHA DAMASCONE | 24720-09-0 | 1-(2,6,6-TRIMETHYLCYCLOHEX-2-EN-1- |
| | | YL)BUT-2-EN-1-ONE |
| APRITONE | 68133-79-9 | 2-(3,7-DIMETHYLOCTA-2,6-DIEN-1- |
| | | YL)CYCLOPENTAN-1-ONE |
| BAMBOO KETONE | 113486-29-6 | 3-METHYLNONANE-2,4-DIONE |
| BENZALDEHYDE | 100-52-7 | BENZALDEHYDE |
| BENZYL ALCOHOL | 100-51-6 | PHENYLMETHANOL |
| BETA GAMMA HEXENOL | 928-96-1 | HEX-3-EN-1-OL |
| BUCCOXIME | 75147-23-8 | 1,5-DIMETHYLBICYCLO[3.2.1]OCTAN-8- |
| | | ONE OXIME |
| CALONE | 28940-11-6 | 7-METHYL-2H-BENZO[B][1,4]DIOXEPIN- |
| | | 3(4H)-ONE |
| CANTHOXAL | 5462-06-6 | 3-(4-METHOXYPHENYL)-2- |
| | | METHYLPROPANAL |
| CASSIFFIX | 139539-66-5 | 5-METHYL-1-(2,2,3- |
| | | TRIMETHYLCYCLOPENT-3-EN-1-YL)-6- |
| | | OXABICYCLO[3.2.1]OCTANE |
| CASSYRANE | 871465-49-5 | 2-(TERT-BUTYL)-5-METHYL-2-PROPYL- |
| | | 2,5-DIHYDROFURAN |
| CIS JASMONE | 488-10-8 | 3-METHYL-2-(PENT-2-EN-1- |
| | | YL)CYCLOPENT-2-EN-1-ONE |
| CIS-6-NONEN-1-OL | 35854-86-5 | NON-6-EN-1-OL |
| CITRONELLYL | 7492-67-3 | 2-((3,7-DIMETHYLOCT-6-EN-1- |
| OXYACETALDEHYDE | | YL)OXY)ACETALDEHYDE |
| CITRUS OIL DIST 2600 | 68647-72-3 | TERPENES AND TERPENOIDS, SWEET |
| | | ORANGE-OIL |
| CYCLEMAX | 7775-00-0 | 3-(4-ISOPROPYLPHENYL)PROPANAL |
| CYCLOPENTOL | 84560-00-9 | 2-PENTYLCYCLOPENTAN-1-OL |
| DAMASCENONE | 23696-85-7 | 1-(2,6,6-TRIMETHYLCYCLOHEXA-1,3- |
| | | DIEN-1-YL)BUT-2-EN-1-ONE |
| DAMASCONE BETA | 23726-91-2 | 1-(2,6,6-TRIMETHYLCYCLOHEX-1-EN-1- |
| | | YL)BUT-2-EN-1-ONE |
| DELPHONE | 4819-67-4 | 2-PENTYLCYCLOPENTAN-1-ONE |
| DELTA DAMASCONE | 57378-68-4 | 1-(2,6,6-TRIMETHYLCYCLOHEX-3-EN-1- |
| | | YL)BUT-2-EN-1-ONE |
| DIHYDRO-BETA-IONONE | 17283-81-7 | 4-(2,6,6-TRIMETHYLCYCLOHEX-1-EN-1- |
| | | YL)BUTAN-2-ONE |
| DIMETHYL SULFIDE | 75-18-3 | DIMETHYLSULFANE |
| EBELIA | 1253785-23-7 | 1-METHOXY-7,9-DIMETHYL-2- |
| | | OXASPIRO[5.5]UNDEC-8-ENE |
| ETASPIRENE 940121 | 79893-63-3 | 6-ETHYL-2,10,10-TRIMETHYL-1- |
| | | OXASPIRO[4.5]DECA-3,6-DIENE |
| EZ-26 NONADIEN-1-AL | 557-48-2 | NONA-2,6-DIENAL |
| FLEURAMONE | 137-03-1 | 2-HEPTYLCYCLOPENTAN-1-ONE |
| FLORIDILE | 131379-24-3 | UNDEC-9-ENENITRILE |
| FRAMBINON METHYL | 104-20-1 | 4-(4-METHOXYPHENYL)BUTAN-2-ONE |
| ETHER | | |
| FRUTONILE | 69300-15-8 | 2-METHYLDECANENITRILE |
| FURANEOL | 3658-77-3 | 4-HYDROXY-2,5-DIMETHYLFURAN- |
| | | 3(2H)-ONE |
| GERANYL FORMATE | 105-86-2 | 3,7-DIMETHYLOCTA-2,6-DIEN-1-YL |
| | | FORMATE |
| HELIOTROPIN | 120-57-0 | BENZO[D][1,3]DIOXOLE-5- |
| | | CARBALDEHYDE |
| HERBANATE | 116126-82-0 | ETHYL 3- |
| | | ISOPROPYLBICYCLO[2.2.1]HEPT-5-ENE- |
| | | 2-CARBOXYLATE |
| HEXALON | 79-78-7 | 1-(2,6,6-TRIMETHYLCYCLOHEX-2-EN-1- |
| | | YL)HEPTA-1,6-DIEN-3-ONE |
| HEXANAL | 66-25-1 | HEXANAL |
| IONONE BETA | 14901-07-6 | 4-(2,6,6-TRIMETHYLCYCLOHEX-1-EN-1- |
| | | YL)BUT-3-EN-2-ONE |
| IONONE GAMMA METHYL | 127-51-5 | 3-METHYL-4-(2,6,6- |
| | | TRIMETHYLCYCLOHEX-2-EN-1- |
| | | YL)BUT-3-EN-2-ONE |
| LABIENOXIME | 81783-01-9 | 2,4,4,7-TETRAMETHYLNONA-6,8-DIEN- |
| | | 3-ONE OXIME |
| MELONAL | 106-72-9 | 2,6-DIMETHYLHEPT-5-ENAL |
| METHOXY MELONAL | 62439-41-2 | 6-METHOXY-2,6-DIMETHYLHEPTANAL |
| METHYL AMYL KETONE | 110-43-0 | HEPTAN-2-ONE |
| METHYL HEPTENONE | 110-93-0 | 6-METHYLHEPT-5-EN-2-ONE |
| METHYL HEXYL KETONE | 111-13-7 | OCTAN-2-ONE |
| METHYL NONYL KETONE | 112-12-9 | UNDECAN-2-ONE |
| NECTARYL | 95962-14-4 | 2-(2-(4-METHYLCYCLOHEX-3-EN-1- |
| | | YL)PROPYL)CYCLOPENTAN-1-ONE |
| NEOCASPIRENE EXTRA | 89079-92-5 | 10-ISOPROPYL-2,7-DIMETHYL-1- |
| | | OXASPIRO[4.5]DECA-3,6-DIENE |
| OCTALYNOL | 103614-86-4 | 2,2,6,8-TETRAMETHYL-1,2,3,4,4A,5,8,8A- |
| | | OCTAHYDRONAPHTHALEN-1-OL |
| OCTYL ALCOHOL | 111-87-5 | OCTAN-1-OL |
| OSMANTOL NONTSCA | 2209852-19-5 | 3,5-DIMETHYLOCT-5-EN-4-OL |
| 969325 | | |
| OXANE | 59323-76-1 | 2-METHYL-4-PROPYL-1,3-OXATHIANE |
| PARA HYDROXY PHENYL | 5471-51-2 | 4-(4-HYDROXYPHENYL)BUTAN-2-ONE |
| BUTANONE | | |
| PARA METHYL | 122-00-9 | 1-(P-TOLYL)ETHAN-1-ONE |
| ACETOPHENONE | | |
| PARADISAMIDE | 406488-30-0 | 2-ETHYL-N-METHYL-N-(M- |
| | | TOLYL)BUTANAMIDE |
| PETALIA | 916887-53-1 | ALPHA-CYCLOHEXYLIDENE-2- |
| | | METHYL-BENZENEACETONITRILE |
| POMAROSE | 357650-26-1 | 5,6,7-TRIMETHYLOCTA-2,5-DIEN-4-ONE |
| RHUBOFLOR | 93939-86-7 | 6-ETHYLIDENEOCTAHYDRO-2H-5,8- |
| | | METHANOCHROMENE |
| RINGONOL | 38462-22-5 | 2-(2-MERCAPTOPROPAN-2-YL)-5- |
| | | METHYLCYCLOHEXAN-1-ONE |
| SAUVIGNONE 100 | 851768-51-9 | 5-MERCAPTO-5-METHYLHEXAN-3-ONE |
| STRAWBERIFF | 3142-72-1 | 2-METHYLPENT-2-ENOIC ACID |
| STRAWBINONE | 4643-27-0 | OCT-2-EN-4-ONE |
| VELOUTONE | 65443-14-3 | 2,2,5-TRIMETHYL-5- |
| | | PENTYLCYCLOPENTAN-1-ONE |
| VERTOFRUCT EU | 54889-63-3 | 1,4- |
| | | BIS(ETHOXYMETHYL)CYCLOHEXANE |

It may be particularly preferred to control the weight ratio of the Table 1 fragrance material to its counterpart (e.g., fragrance materials listed in Table 2). For example, the weight ratio of the Table 1 fragrance material with fruity notes to the second fragrance material may be from 100:0 to 1:99, or from 100:0 to 25:75, or from 100:0 to 50:50, or from 100:0 to 51:49, or from 100:0 to 75:25, or from 100:0 to 90:10, or from 100:0 to 95:5, or from 100:0 to 99:1.

In order to provide a full and desirable olfactory experience, the fragrance composition may comprise three or more fragrance materials. The fragrance composition may further comprise a third fragrance material. The fragrance composition may comprise from two to about fifty fragrance materials, or from two to about thirty fragrance materials. For simplicity and ease of manufacturing, it may be desirable to limit the number of components of the fragrance composition. For example, the fragrance composition may comprise from two to about twenty, or to about fifteen, or to about ten, fragrance materials.

The fragrance composition may further comprise a carrier, preferably a carrier comprising isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or a mixture thereof, more preferably a carrier comprising isopropyl myristate.

Other useful carriers may be solvents or emulsifiers, such as nonionic surfactants, which may help with incorporation of the fragrance composition into a final product form. The fragrance composition may be in the form of an emulsion, preferably an oil-in-water emulsion.

The fragrance composition of the present disclosure may be incorporated into a consumer product, which are described in more detail above. Thus, the present disclosure relates to a consumer product comprising the fragrance composition as described herein. Preferably, the consumer product is a baby care product, a beauty care product, a fabric care product, a home care product, a family care product, a feminine care product, a health care product, a food product, a beverage product, a fine fragrance product, or a combination thereof.

The fragrance composition may be present in the consumer product in an amount of at least 0.1%, preferably at least 0.5%, more preferably at least 1%, by weight of the consumer product. The fragrance composition may be present in the consumer product in an amount of from about 0.1% to about 99.9%, or from about 0.1% to about 50%, or from about 0.1% to about 25%, or from about 0.1% to about 10%, or from about 0.1% to about 5%, or from about 1% to about 5%, by weight of the consumer product.

### Additional Fragrance Materials:

The fragrance compositions disclosed herein may also include additional fragrance materials. Some non-limiting examples of such additional fragrance materials are listed in Table 3 below.

TABLE 3

| IUPAC NAME | CAS |
|---|---|
| (2-ETHOXYETHYL)(P-TOLYL)SULFANE | 57023-03-7 |
| (2-METHOXYETHYL)(P-TOLYL)SULFANE | 1779128-42-5 |
| (3-(2-METHOXYETHYL)BICYCLO[3.3.1]NONAN-1-YL)METHYL | — |
| ACETATE | |
| (3-ETHYLADAMANTAN-1-YL)METHYL ACETATE | — |
| (3-METHYLADAMANTAN-1-YL)METHYL ACETATE | — |
| (5,6-DIMETHYLBENZO[D]OXAZOL-2-YL)METHANOL | — |
| (5-METHYLBICYCLO[5.3.1]UNDECAN-1-YL)METHYL ACETATE | — |
| (Z)-DODEC-8-EN-1-YL ACETATE | 28079-04-1 |
| 1-(4-ETHYLCYCLOHEXYL)-3,3-DIMETHYLBUTAN-2-ONE | 1515850-30-2 |
| 1-(ETHOXYCARBONYL)CYCLOBUTANE-1-CARBOXYLIC ACID | 54450-84-9 |
| 1-(PENTYLOXY)HEXANE | 32357-83-8 |
| 1,10-BIS(METHYLTHIO)DECANE | 100393-11-1 |
| 1,2-BIS(2,5-DIETHYLFURAN-3-YL)DISULFANE | — |
| 1,2-BIS(2,5-DIMETHYLFURAN-3-YL)DISULFANE | 28588-73-0 |
| 1,2-BIS(2-ETHYL-5-METHYLFURAN-3-YL)DISULFANE | — |
| 1,2-BIS(2-METHYLFURAN-3-YL)DISULFANE | 28588-75-2 |
| 1,8-BIS(METHYLTHIO)OCTANE | 20295-48-1 |
| 1,9-BIS(METHYLTHIO)NONANE | 71522-68-4 |
| 10-ETHOXY-10-OXODECANOIC ACID | 693-55-0 |
| 1-ALLYL-4-ETHYLBENZENE | 18640-62-5 |
| 1-ALLYL-4-METHYLBENZENE | 3333-13-9 |
| 1-BUTOXYHEXANE | 54459-71-1 |
| 1-CYCLOHEXYL-2-ETHYLHEXAN-1-ONE | 50395-57-8 |
| 1-CYCLOHEXYL-2-METHYLBUTAN-1-ONE | 17773-60-3 |
| 1-CYCLOHEXYL-2-METHYLPROPAN-1-ONE | 1125-71-9 |
| 1-CYCLOHEXYL-3,3-DIMETHYLBUTAN-2-ONE | 23137-42-0 |
| 1-ETHOXYHEXANE | 5756-43-4 |
| 1-ETHYL 3-METHYL 2-ISOBUTYLMALONATE | 1135264-48-0 |
| 1-PROPOXYHEXANE | 53685-78-2 |
| 2-(5,6-DIMETHYLBENZO[D]OXAZOL-2-YL)ACETONITRILE | 88447-16-9 |
| 2-(CYCLOHEX-1-EN-1-YL)ACETIC ACID | 18294-87-6 |
| 2-(CYCLOOCT-1-EN-1-YL)ACETIC ACID | 56900-24-4 |
| 2-(ETHOXYCARBONYL)-4-METHYLPENTANOIC ACID | 2985-36-6 |
| 2-(P-TOLYLTHIO)ETHAN-1-OL | 13290-16-9 |
| 2,2,5-TRIMETHYL-5-PENTYLCYCLOPENTAN-1-ONE | 65443-14-3 |
| 2,2,6,6,8,8-HEXAMETHYLNONAN-4-ONE | — |
| 2,2-DIMETHYL-5-PROPYLCYCLOPENTAN-1-ONE | 325168-16-9 |
| 2,5,6-TRIMETHYLBENZO[D]OXAZOLE | 19219-98-8 |
| 2-BUTYL-2,5,5-TRIMETHYLCYCLOPENTAN-1-ONE | 65443-15-4 |
| 2-BUTYLCYCLOHEXAN-1-ONE | 1126-18-7 |
| 2-BUTYLOCTYL BUTYRATE | 1007089-49-7 |
| 2-ETHYLCYCLOHEXAN-1-ONE | 4423-94-3 |
| 2-ETHYLHEPTYL BUTYRATE | 94200-05-2 |
| 2-ETHYLHEXYL BUTYRATE | 25415-84-3 |
| 2-ETHYLHEXYL PROPIONATE | 6293-37-4 |
| 2-HEXYL-2,5,5-TRIMETHYLCYCLOPENTAN-1-ONE | 65443-16-5 |
| 2-HYDROXYETHYL 2-AZABICYCLO[2.2.1]HEPTANE-2- | 1517175-51-7 |
| CARBOXYLATE | |
| 2-METHYL-2,5,5-TRIPROPYLCYCLOPENTAN-1-ONE | — |
| 2-METHYL-5-(PROP-2-YN-1-YLOXY)BENZO[D]THIAZOLE | 64225-41-8 |
| 2-METHYL-5-PROPOXYBENZO[D]THIAZOLE | 1799562-88-1 |
| 2-METHYLBENZO[D]THIAZOLE | 120-75-2 |
| 2-METHYLNAPHTHO[2,1-D]OXAZOLE | 20686-65-1 |
| 2-NONYLCYCLOHEXAN-1-ONE | 16556-72-2 |
| 2-PENTYLCYCLOHEXAN-1-ONE | 32362-97-3 |
| 2-PROPYLCYCLOHEXAN-1-ONE | 94-65-5 |
| 2-PROPYLHEPTYL BUTYRATE | 908822-62-8 |
| 3-(FORMYLOXY)BUTYL ACETATE | 344354-26-3 |
| 3,3′,5,5′-TETRAMETHYL-1,1′-BIPHENYL | 25570-02-9 |
| 3,3,5,5-TETRAMETHYLCYCLOHEXAN-1-ONE | 14376-79-5 |
| 3,3,5-TRIMETHYLCYCLOHEXAN-1-ONE | 873-94-9 |
| 3,3-DIMETHYL-1-(4-METHYLCYCLOHEXYL)BUTAN-2-ONE | 1513612-25-3 |
| 3,3-DIMETHYL-1-(4-PROPYLCYCLOHEXYL)BUTAN-2-ONE | — |
| 3,3′-DIMETHYL-1,1′-BIPHENYL | 612-75-9 |
| 3,3-DIMETHYLCYCLOHEXAN-1-ONE | 2979-19-3 |
| 3,4′-DIMETHYL-1,1′-BIPHENYL | 7383-90-6 |
| 3,5-DIETHYLBENZALDEHYDE | 81698-95-5 |
| 3,5-DIMETHYL-1-(P-TOLYL)-1H-PYRAZOLE | 20157-46-4 |
| 3,5-DIMETHYL-1,1′-BIPHENYL | 17057-88-4 |
| 3,5-DIMETHYL-1-PHENYL-1H-PYRAZOLE | 1131-16-4 |
| 3A,4,5,6,7,7A-HEXAHYDRO-1H-4,7-METHANOINDEN-6-YL 2- | 138579-56-3 |
| METHYLBUTANOATE | |
| 3A,4,5,6,7,7A-HEXAHYDRO-1H-4,7-METHANOINDEN-6-YL | 5413-60-5 |
| ACETATE | |
| 3A,4,5,6,7,7A-HEXAHYDRO-1H-4,7-METHANOINDEN-6-YL | 17511-61-4 |
| BUTYRATE | |
| 3A,4,5,6,7,7A-HEXAHYDRO-1H-4,7-METHANOINDEN-6-YL | 68039-39-4 |
| ISOBUTYRATE | |
| 3A,4,5,6,7,7A-HEXAHYDRO-1H-4,7-METHANOINDEN-6-YL | 17511-60-3 |
| PROPIONATE | |
| 3-DISULFANEYL-2,5-DIMETHYLFURAN | |
| 3-ETHOXYBUTYL ACETATE | 1118987-13-5 |
| 3-ETHYL-5-METHYLBENZALDEHYDE | 3132-93-2 |
| 3-ETHYLBENZALDEHYDE | 34246-54-3 |
| 3-ISOPROPOXYBUTYL ACETATE | — |
| 3-ISOPROPYLBENZALDEHYDE | 34246-57-6 |
| 3-METHOXYBUTYL ACETATE | 4435-53-4 |
| 3-METHYL-1,1′-BIPHENYL | 643-93-6 |
| 3-PHENYLPROP-1-EN-1-ONE | 87101-44-8 |
| 3-PROPOXYBUTYL ACETATE | — |
| 4-(3,5-DIMETHYL-1H-PYRAZOL-1-YL)ANILINE | 52708-32-4 |
| 4-(3,5-DIMETHYL-1H-PYRAZOL-1-YL)PHENOL | 51011-27-9 |
| 4-(CYCLOPROPANECARBONYL)PHENYL FORMATE | — |
| 4-(HEPTYLOXY)BENZALDEHYDE | 27893-41-0 |
| 4-(HEXYLOXY)BENZALDEHYDE | 5736-94-7 |
| 4-(METHOXYCARBONYL)BICYCLO[2.2.1]HEPTANE-1- | 15448-77-8 |
| CARBOXYLIC ACID | |
| 4-(METHOXYCARBONYL)CYCLOHEXANE-1-CARBOXYLIC ACID | 32529-79-6 |
| 4-(PENTYLOXY)BENZALDEHYDE | 5736-91-4 |
| 4-FORMYLPHENYL 3-METHYLBUTANOATE | 1177233-18-9 |
| 4-FORMYLPHENYL BUTYRATE | 50262-49-2 |
| 4-FORMYLPHENYL PENT-4-YNOATE | 2056250-88-3 |
| 4-FORMYLPHENYL PENTANOATE | 50262-50-5 |
| 4-METHOXYBICYCLO[2.2.2]OCTANE-1-CARBOXYLIC ACID | 773-34-2 |
| 5,10-DIMETHYLUNDECAN-2-ONE | 55105-48-1 |
| 5,5,7,7-TETRAMETHYLOCTAN-3-ONE | — |
| 5,6-DIMETHYLBENZO[D]OXAZOLE-2-CARBONITRILE | 2468436-52-2 |
| 5,9-DIMETHYLDECAN-2-ONE | 33933-82-3 |
| 5-ETHOXY-2-METHYLBENZO[D]THIAZOLE | 123771-99-3 |
| 5-ETHYLISOPHTHALALDEHYDE | 118792-76-0 |
| 5-METHOXY-2-METHYLBENZO[D]THIAZOLE | 2941-69-7 |
| 5-METHYLDECAN-2-ONE | 56608-65-2 |
| 5-METHYLNONAN-2-ONE | 2358176-40-4 |
| 5-METHYLOCTAN-2-ONE | 58654-67-4 |
| 6-(ALLYLOXY)HEXYL ACRYLATE | — |
| 6-(FORMYLOXY)HEXYL ACRYLATE | — |
| 6-(FORMYLOXY)HEXYL METHACRYLATE | — |
| 6-(HEXYLOXY)HEXYL METHACRYLATE | — |
| 6-ACETOXYHEXYL METHACRYLATE | 116767-82-9 |
| 6-METHOXYHEXYL ACRYLATE | 106128-35-2 |
| ADAMANTAN-1-YLMETHYL ACETATE | 778-11-0 |
| ALLYLBENZENE | 300-57-2 |
| BUT-2-EN-1-YLBENZENE | 1560-06-1 |
| CYCLOHEPTYL CYCLOHEXANECARBOXYLATE | — |
| CYCLOHEXYL 2-ETHYLHEXANOATE | 176523-52-7 |
| CYCLOHEXYL 4-METHYLCYCLOHEXANE-1-CARBOXYLATE | 77954-32-6 |
| CYCLOHEXYL CYCLOHEXANECARBOXYLATE | 15840-96-7 |
| CYCLOHEXYL(CYCLOOCTYL)METHANONE | 1522600-44-7 |
| CYCLOPROPYL(4-ETHOXYPHENYL)METHANONE | 75343-44-1 |
| CYCLOPROPYL(4-ISOPROPOXYPHENYL)METHANONE | 1152833-63-0 |
| CYCLOPROPYL(4-METHOXYPHENYL)METHANONE | 7152-03-6 |
| CYCLOPROPYL(4-PROPOXYPHENYL)METHANONE | 1226182-72-4 |
| DIBUTYL 2-ISOBUTYLMALONATE | 101083-45-8 |
| DIBUTYL CYCLOBUTANE-1,1-DICARBOXYLATE | 98899-26-4 |
| DICYCLOHEXYLMETHANONE | 119-60-8 |
| DIETHYL 2-ISOBUTYLMALONATE | 10203-58-4 |
| DIETHYL CYCLOBUTANE-1,1-DICARBOXYLATE | 3779-29-1 |
| DIETHYL DECANEDIOATE | 110-40-7 |
| DIMETHYL BICYCLO[2.2.1]HEPTANE-1,4-DICARBOXYLATE | 15448-76-7 |
| DIMETHYL CYCLOHEXANE-1,4-DICARBOXYLATE | 94-60-0 |
| DIPENTYL CYCLOBUTANE-1,1-DICARBOXYLATE | — |
| DIPROPYL 2-ISOBUTYLMALONATE | — |
| DIPROPYL CYCLOBUTANE-1,1-DICARBOXYLATE | 1823295-70-0 |
| DODECYL(METHYL)SULFANE | 3698-89-3 |
| ETHYL 10-OXODECANOATE | 692-87-5 |
| ETHYL 2-(1-HYDROXY-2-METHYLCYCLOHEXYL)ACETATE | 5108-87-2 |
| ETHYL 2-(2-HYDROXYADAMANTAN-2-YL)ACETATE | 112181-97-2 |
| ETHYL 2-AZABICYCLO[2.2.1]HEPTANE-2-CARBOXYLATE | 910857-00-0 |
| ETHYL 3-(4-(METHOXYMETHOXY)PHENYL)-3-OXOPROPANOATE | 178609-50-2 |
| ETHYL 3-(4-ETHOXYPHENYL)-3-OXOPROPANOATE | 51725-83-8 |
| ETHYL 3-(4-METHOXYPHENYL)-3-OXOPROPANOATE | 2881-83-6 |
| ETHYL 3-CYCLOHEXYL-3-HYDROXYBUTANOATE | 28811-84-9 |
| ETHYL 3-HYDROXY-3,4-DIMETHYLOCTANOATE | 65790-83-2 |
| ETHYL 4-METHOXY-1,4-DIMETHYLCYCLOHEXANE-1- | — |
| CARBOXYLATE | |
| ETHYL 4-METHOXYBICYCLO[2.2.2]OCTANE-1-CARBOXYLATE | 96454-88-5 |
| ETHYL N-HYDROXYETHANIMIDOTHIOATE | 20639-32-1 |
| ETHYL OCTANOATE | 106-32-1 |
| ETHYL(P-TOLYL)SULFANE | 622-63-9 |
| HEPTADECYL(METHYL)SULFANE | 62155-11-7 |
| HEXANE-1,6-DIYL DIACRYLATE | 13048-33-4 |
| HEXYL ACRYLATE | 2499-95-8 |
| HEXYL METHACRYLATE | 142-09-6 |
| ISOPROPYL BICYCLO[2.2.1]HEPT-5-ENE-2-CARBOXYLATE | 916824-62-9 |
| METHYL 3-(4-METHOXYPHENYL)-3-OXOPROPANOATE | 22027-50-5 |
| METHYL 3,5-DIHYDROXYADAMANTANE-1-CARBOXYLATE | 83406-10-4 |
| METHYL 3-HYDROXY-5,7-DIMETHYLADAMANTANE-1- | — |
| CARBOXYLATE | |
| METHYL 3-HYDROXY-5-METHYLADAMANTANE-1- | — |
| CARBOXYLATE | |
| METHYL 3-HYDROXYADAMANTANE-1-CARBOXYLATE | 68435-07-4 |
| METHYL 4-(HYDROXYMETHYL)BICYCLO[2.2.1]HEPTANE-1- | 1350821-95-2 |
| CARBOXYLATE | |
| METHYL 4-(HYDROXYMETHYL)CYCLOHEXANE-1- | 13380-85-3 |
| CARBOXYLATE | |
| METHYL 4-(METHOXYMETHYL)BICYCLO[2.2.1]HEPTANE-1- | 2725791-10-4 |
| CARBOXYLATE | |
| METHYL 4-FORMYLCYCLOHEXANE-1-CARBOXYLATE | 37942-76-0 |
| METHYL 4-HYDROXYBICYCLO[2.2.2]OCTANE-1-CARBOXYLATE | 23062-53-5 |
| METHYL 4-METHOXYBICYCLO[2.2.2]OCTANE-1-CARBOXYLATE | 81687-90-3 |
| METHYL N-HYDROXYETHANIMIDOTHIOATE | 13749-94-5 |
| OXEPANE | 592-90-5 |
| TERT-BUTYL (1S,4R)-2-AZABICYCLO[2.2.1]HEPTANE-2- | 1932009-15-8 |
| CARBOXYLATE | |
| TERT-BUTYL 2-AZABICYCLO[2.2.1]HEPTANE-2-CARBOXYLATE | 1495791-95-1 |
| TERT-BUTYL 2-ETHYL-4-METHYLPYRROLIDINE-1- | 1934925-19-5 |
| CARBOXYLATE | |
| TERT-BUTYL 3-METHYLBICYCLO[2.2.1]HEPT-5-ENE-2- | — |
| CARBOXYLATE | |
| TERT-BUTYL 6-METHYL-2-AZABICYCLO[2.2.1]HEPTANE-2- | — |
| CARBOXYLATE | |
| TERT-BUTYL BICYCLO[2.2.1]HEPT-5-ENE-2-CARBOXYLATE | 154970-45-3 |
| TERT-PENTYL BICYCLO[2.2.1]HEPT-5-ENE-2-CARBOXYLATE | 398140-58-4 |
| TETRADEC-12-EN-1-YL ACETATE | 35153-21-0 |
| THIANTHRENE | 92-85-3 |
| TRIDEC-11-EN-1-YL ACETATE | 33951-95-0 |

### Process of Making a Consumer Product

The present disclosure also relates to processes of making a consumer product. For example, the process may comprise the steps of: combining a fragrance material with a treatment adjunct or a substrate, where the fragrance material is an ester containing fragrance material with fruity notes (e.g., one or more of the fragrance materials detailed in Table 1).

Preferably, the fragrance material is in liquid form. The fragrance material may be combined with one or more other materials. For example, the fragrance material may be part of a fragrance composition that comprises at least one fragrance material from Table 2, or another fragrance material that is useful herein.

The fragrance material may be part of a fragrance composition, which may be combined with a treatment adjunct to form a consumer product.

The consumer products of the present disclosure can be formulated into any suitable form and prepared by any process chosen by the formulator. The fragrance materials, second fragrance materials (if any), fragrance compositions, treatment adjuncts, and/or base compositions may be combined in a batch process, in a circulation loop process, and/or by an in-line mixing process. Suitable equipment for use in the processes disclosed herein may include continuous stirred tank reactors, homogenizers, turbine agitators, recirculating pumps, paddle mixers, high shear mixers, static mixers, plough shear mixers, ribbon blenders, vertical axis granulators and drum mixers, both in batch and, where available, in continuous process configurations, spray dryers, and extruders.

The resulting compositions may be placed into a container to form a consumer product, as described herein. The container may be a bottle, preferably a plastic bottle, more preferably a plastic bottle comprising recycled plastic. The resulting composition may be placed into an aerosol or other spray container according to known methods.

The process may comprise the steps of combining a fragrance material with a substrate. The substrate may be a non-woven sheet. The substrate may be an absorbent material or article. The first fragrance may be combined with the substrate by any suitable method, including spraying.

### Process of Treatment

The present disclosure also relates to a process of treating a surface or an environment with a fragrance material with fruity notes as detailed herein. The fragrance material with fruity notes may be part of a consumer product.

The process of treatment comprises the step of providing the fragrance material with fruity notes to a surface or environment. Suitable surfaces may include fabric, hard surfaces, dishware, hair, or skin. Suitable environments may include interior spaces such as a room in a residential dwelling, a commercial property, or an industrial setting.

The process may include the step of contacting a surface, preferably a fabric, with the fragrance material with fruity notes, optionally as part of a consumer product according to the present disclosure.

The process may include the step of providing the fragrance material with fruity notes to an environment, preferably an interior environment, optionally as part of a consumer product according to the present disclosure. The step of providing the fragrance material with fruity notes may occur via a spraying process or through diffusion.

### Use

The present disclosure also relates to the use of a fragrance material with fruity notes, to provide a pleasing fragrance to a consumer product, a surface, or an environment. Such materials, products, surfaces, and environments are discussed in more detail above.

### Combinations

Specifically contemplated combinations of the disclosure are herein described in the following lettered paragraphs. These combinations are intended to be illustrative in nature and are not intended to be limiting.
A. A consumer product comprising a fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate; and b) a treatment adjunct; and wherein the fragrance material is present in an amount of between about 0.0005% and about 0.8%, by weight of the consumer product composition.
B. A consumer product according to Paragraph A, wherein the fragrance material is present in the consumer product in an amount of at least 0.001%, preferably at least 0.01%, more preferably at least 0.1%, more preferably at least 0.5%, more preferably at least 0.75%, by weight of the consumer product.
C. A consumer product according to any of Paragraphs A-B, wherein the fragrance material is part of a perfume delivery system, where the perfume delivery system is a delivery particle, a pro-fragrance material, or a combination thereof.
D. A consumer product according to any of Paragraphs A-C, wherein the fragrance material is part of a perfume delivery system, where the perfume delivery system is a delivery particle, a pro-fragrance material, or a combination thereof.
E. A consumer product according to any of Paragraphs A-D, wherein the treatment adjunct is selected from a group consisting of: surfactants, conditioning actives, deposition aids, rheology modifiers, structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, or mixtures thereof.
F. A consumer product according to any of Paragraphs A-E, wherein the treatment adjunct comprises a carrier, preferably a carrier comprising isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or a mixture thereof, more preferably a carrier comprising isopropyl myristate.
G. A consumer product according to any of Paragraphs A-F, wherein the consumer product is a baby care product, a beauty care product, a fabric care product, a home care product, a family care product, a feminine care product, a health care product, a food product, a beverage product, a fine fragrance product, or a combination thereof.
H. A consumer product according to any of Paragraphs A-G, wherein the consumer product is a fabric care product and/or a home care product, wherein the home care product is selected from the group consisting of a hard surface cleaner, a dish care product, an air care product, or a combination thereof.
I. A consumer product according to any of Paragraphs A-H, wherein the consumer product comprises a form selected from a liquid composition, a granular composition, a hydrocolloid, a single-compartment pouch, a multi-compartment pouch, a dissolvable sheet, a pastille or bead, a fibrous article, a tablet, a stick, a bar, a flake, a spray, a foam/mousse, a non-woven sheet, a cream, an absorbent article, a paste, a gel, an adhesive, an adhesive patch, or a mixture thereof.
J. A process of making a consumer product, the process comprising the steps of combining a fragrance material with a treatment adjunct or a substrate, wherein the fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.
K. A consumer product according to Paragraph J, wherein the fragrance material is in liquid form.
L. A process of treating a surface or an environment, the process comprising the step of providing a fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, to a surface or environment.
M. A use of fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.
N. A consumer product comprising a first fragrance material comprising at least one of: hexyl methacrylate, diethyl isobutylmalonate, diethyl cyclobutane-1,1-dicarboxylate and methyl 4-methoxybicyclo[2,2,2]octane-1-carboxylate; and a treatment adjunct; wherein the first fragrance material is present in an amount of between about 0.0003% and about 20.0%, by weight of the consumer product composition.
O. A consumer product according to Paragraph N, wherein the fragrance material is present in the consumer product in an amount of at least 1.0%, preferably at least 2.0%, more preferably at least 5.0%, more preferably at least 10.0%, more preferably at least 15.0%, by weight of the consumer product.
P. A consumer product according to any of Paragraphs N-O, wherein the fragrance material is part of a perfume delivery system, where the perfume delivery system is a delivery particle, a pro-fragrance material, or a combination thereof.
Q. A consumer product according to any of Paragraphs N-P, wherein the fragrance material is part of a perfume delivery system, where the perfume delivery system is a delivery particle, a pro-fragrance material, or a combination thereof.
R. A consumer product according to any of Paragraphs N-Q, wherein the treatment adjunct comprises a carrier, preferably a carrier comprising isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or a mixture thereof, more preferably a carrier comprising isopropyl myristate.
S. A consumer product according to any of Paragraphs N-R, wherein the consumer product is an air care product, a fine fragrance product, or a combination thereof.

### Test Methods

It is understood that the test methods disclosed in the Test Methods section of the present application should be used to determine the respective values of the parameters of Applicant's claimed subject matter as claimed and described herein.

### Perfume Headspace Method

Perfume headspace data is collected for various materials according to the method provided below.

The partition coefficients are calculated by determining the amount of PRM in the headspace above a solution with a known concentration using a GC/FID-MS (Agilent 7890 and 5977A MSD) with a thermal desorption unit (Gerstel MPS2) and Dynamic Headspace Sampler (Gerstel DHS). This technique allows for automated headspace collections using the MultiPurposeSampler (MPS). The PRMs of interest are prepared at a known concentration in isopropyl myristate (IPM) or propylene glycol monomethyl ether (DPM), and 1 mL is pipetted in a 20 mL headspace vial.

DHS Conditions: The sample is tempered in the unit at 30° C. A helium stream at 10 mls/min purges 20 mL of headspace containing the analytes above the sample onto the sorbent tube (TDU) containing 25 mg of TenaxTA 35/60 (Restek cat #25701).

TDU conditions: The sorbent tube is desorbed with a program starting at 30° C. solvent vent for 2 min then ramped at 720° C./min to 270° C. and held for 8 min. The analytes are refocused on a glass bead liner at −60° C. then desorbed at a rate of 12° C./s to 270° C. onto the GC column.

GC/MS conditions: Agilent 122-5532UI column of 30 m×250 μm×0.25 μm and a flow rate of 1.5 mL/min is utilized. A temperature program of 50° C. to 280° C. with a 2 min hold and a ramp rate of 12° C./min is suitable. Scan or SIM mode MSD parameters with a solvent delay for the DPM are applied to achieve appropriate detection.

Analytical standards can be obtained from AK Scientific, Inc., Union City, CA 94587, USA. A stock solution is prepared at approximately 0.5 g/L. The stock solution is then diluted to 4 to 5 concentrations ranging from approximately 0.05 g/L to 0.00005 g/L. These standard solutions are then analyzed by GC/MS. This analysis is performed by using the same TDU desorption and GC/MS conditions described above with a 1 uL liquid injection onto the TDU tube instead of the DHS collection. The amount injected on column and the area responses of the target m/z are utilized to create a calibration plot with a linear fit.

Quantification of the headspace is performed by the PRM target m/z area response of the perfume component and the linear equation of the calibration plot.

### EXAMPLES

The examples provided below are intended to be illustrative in nature and are not intended to be limiting.

### Example 1. Headspace Data

Perfume headspace data was collected for 3-methoxybutyl acetate, hexyl methacrylate, diethyl isobutylmalonate and methyl 4-methoxybicyclo[2,2,2]octane-1-carboxylate in isopropyl myristate (IPM) according to the Headspace Method provided above in the Test Methods section. Perfume headspace data was also collected for Fructalate (Diethyl 1,4-cyclohexanedicarboxylate, CAS No. 72903-27-6) and Pivacyclene (Propanoic acid, 2,2-dimethyl-3a,4,5,6,7,7aa-hexahydro-4,7-methano-1H-inden-6-yl ester; CAS No. 68039-44-1) in isopropyl myristate (IPM) according to the Headspace Method provided above in the Test Methods section. For all the perfume headspace data, the average was recorded after running three replicates.

The perfume headspace data for 3-methoxybutyl acetate, hexyl methacrylate, diethyl isobutylmalonate and methyl 4-methoxybicyclo[2,2,2]octane-1-carboxylate in isopropyl myristate (IPM) was compared against the perfume headspace data for Fructalate and Pivacyclene in isopropyl myristate (IPM). The perfume headspace data ratios are shown below in Table 4. As an example, the Ratio comparing 3-methoxybutyl acetate to Fructalate is:

$$HSR = {HS}_{target} / {HS}_{reference} = 126.4$$

Where HSR is the Head Space Ratio, HS_{target} is the head space of 3-methoxybutyl acetate, and HS_{reference} is the head space of Fructalate.

TABLE 4

| | Head Space Ratio | Head Space Ratio |
|---|---|---|
| | in Comparison | in Comparison |
| Sample | to Fructalate | to Pivacyclene |
| 3-methoxybutyl acetate | 126.4 | 113.6 |
| hexyl methacrylate | 42.4 | 38.1 |
| Diethyl isobutylmalonate | 6.4 | 5.8 |
| Diethyl cyclobutane-1,1- | 26.1 | 10.8 |
| dicarboxylate | | |
| Methyl 4-methoxybicyclo | 1.6 | 1.4 |
| [2.2.2]octane-1-carboxylate | | |

Fructalate and Pivacyclene were chosen for comparison because they were popular fruity esters that were predicted to have a lower vapor pressure than our PRMs. Therefore, the actual test data confirms what was predicted by the model data, i.e., that 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate are more likely to be perceived at lower levels in consumer products, or at greater intensity at equal levels in consumer products, than most other perfume materials with esters that have fruity notes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product comprising:
a) a first fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate; and
b) a treatment adjunct; and
wherein the first fragrance material is present in an amount of between about 0.0003% and about 0.8%, by weight of the consumer product composition.

2. The consumer product according to claim 1, wherein the fragrance material is present in the consumer product in an amount of at least 0.001%, preferably at least 0.01%, more preferably at least 0.1%, more preferably at least 0.5%, more preferably at least 0.75%, by weight of the consumer product.

3. The consumer product according to claim 1, wherein the fragrance material is part of a perfume delivery system, where the perfume delivery system is a delivery particle, a pro-fragrance material, or a combination thereof.

4. The consumer product according to claim 1, wherein the fragrance material is part of a perfume delivery system, where the perfume delivery system is a delivery particle, a pro-fragrance material, or a combination thereof.

5. The consumer product according to claim 1, wherein the treatment adjunct is selected from a group consisting of: surfactants, conditioning actives, deposition aids, rheology modifiers, structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, or mixtures thereof.

6. The consumer product according to claim 1, wherein the treatment adjunct comprises a carrier, preferably a carrier comprising isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or a mixture thereof, more preferably a carrier comprising isopropyl myristate.

7. The consumer product according to claim 1, wherein the consumer product is a baby care product, a beauty care product, a fabric care product, a home care product, a family care product, a feminine care product, a health care product, a food product, a beverage product, a fine fragrance product, or a combination thereof.

8. The consumer product according to claim 1, wherein the consumer product is a fabric care product and/or a home care product, wherein the home care product is selected from the group consisting of a hard surface cleaner, a dish care product, an air care product, or a combination thereof.

9. The consumer product according to claim 1, wherein the consumer product comprises a form selected from a liquid composition, a granular composition, a hydrocolloid, a single-compartment pouch, a multi-compartment pouch, a dissolvable sheet, a pastille or bead, a fibrous article, a tablet, a stick, a bar, a flake, a spray, a foam/mousse, a non-woven sheet, a cream, an absorbent article, a paste, a gel, an adhesive, an adhesive patch, or a mixture thereof.

10. A process of making a consumer product, the process comprising the steps of:
combining a fragrance material with a treatment adjunct or a substrate,
wherein the fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.

11. The process according to claim 10, wherein the fragrance material is in liquid form.

12. A process of treating a surface or an environment, the process comprising the step of providing a fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate, to a surface or environment.

13. A use of fragrance material comprising at least one of: 3-methoxybutyl acetate, hexyl methacrylate, diethyl isobutylmalonate, diethyl cyclobutane-1,1-dicarboxylate and methyl 4-methoxybicyclo[2,2,2]octane-1-carboxylate.

14. A consumer product comprising:
b) a first fragrance material comprising at least one of: hexyl methacrylate, diethyl isobutylmalonate, diethyl cyclobutane-1,1-dicarboxylate and methyl 4-methoxybicyclo[2,2,2]octane-1-carboxylate; and
b) a treatment adjunct; and
wherein the first fragrance material is present in an amount of between about 0.0003% and about 20.0%, by weight of the consumer product composition.

15. The consumer product according to claim 14, wherein the fragrance material is present in the consumer product in an amount of at least 1.0%, preferably at least 2.0%, more preferably at least 5.0%, more preferably at least 10.0%, more preferably at least 15.0%, by weight of the consumer product.

16. The consumer product according to claim 14, wherein the fragrance material is part of a perfume delivery system, where the perfume delivery system is a delivery particle, a pro-fragrance material, or a combination thereof.

17. The consumer product according to claim 14, wherein the fragrance material is part of a perfume delivery system, where the perfume delivery system is a delivery particle, a pro-fragrance material, or a combination thereof.

18. The consumer product according to claim 14, wherein the treatment adjunct comprises a carrier, preferably a carrier comprising isopropyl myristate (IPM), Neobee® caprylic/capric triglyceride, dipropylene glycol, propylene glycol monomethyl ether (DPM), tripropyleneglycol monomethyl ester (TPM), triethyl citrate (TEC), or a mixture thereof, more preferably a carrier comprising isopropyl myristate.

19. The consumer product according to claim 1, wherein the consumer product is an air care product, a fine fragrance product, or a combination thereof.
